(19) United States
(12) Patent Application Publication
(10) Pub. No.: US 2024/0105287 A1
(43) Pub. Date: Mar. 28, 2024

(54) ARTIFICIAL INTELLIGENCE (AI)-BASED SYSTEM AND METHOD FOR PREDICTING PHYSICAL AND CHEMICAL PROPERTIES OF ECO-FRIENDLY MATERIALS

(71) Applicant: Lurong Pan, Hoover, AL (US)

(72) Inventor: Lurong Pan, Hoover, AL (US)

(21) Appl. No.: 17/934,215

(22) Filed: Sep. 22, 2022

(51) Int. Cl. G16C 20/70; C01F 7/02; C01G 15/00; G16C 20/30

(52) U.S. Cl. CPC G16C 20/70; C01F 7/02; C01G 15/00; G16C 20/30

(57) ABSTRACT

A system and method for predicting physical and chemical properties of eco-friendly materials is disclosed. The method includes receiving 3D structures and DFT calculated energy data associated with one or more sesquioxides via one or more electronic devices associated with a user or an external database, and predicting a formation energy and a bandgap energy of each of the one or more sesquioxides based on the received 3D structures and the DFT calculated energy data by using a property prediction-based AI model. Further, the method includes determining a best sesquioxide from the one or more sesquioxides based on the predicted formation energy, and the predicted bandgap energy by using the property prediction-based AI model, and outputting the predicted formation energy, the predicted bandgap energy, and the determined best sesquioxide on user interface screen of the one or more electronic devices associated with the user.

### FIELD OF INVENTION

Embodiments of the present disclosure relate to Artificial Intelligence (AI)-based systems and more particularly relates to an AI-based system and method for predicting physical and chemical properties of eco-friendly materials.

### BACKGROUND

Generally, Aluminum (Al), Gallium (Ga), and Indium (In) sesquioxides are most useful and efficient transparent conductors due to a combination of large bandgap energies and large formation energies. The large bandgap energies show good conductivities and optical transparency over the visible range. Further, the large formation energies show good stability. Furthermore, the Aluminum, Gallium, and Indium sesquioxides are also chemically stable and relatively inexpensive to produce. Thus, predicting formation energy and bandgap energy of sesquioxides is essential to determine stability and conductivity of the sesquioxides respectively. Conventional mechanism of predicting the formation energy and the bandgap energy of sesquioxides is inaccurate resulting in selection of sesquioxides having less stability and less conductivity.

Further, solubility is an important molecular property considered for drug candidates and chemicals. Conventionally, CLogP calculation is used to predict the solubility. However, the CLogP calculation shows a poor prediction of solubility at R²=0.69. Furthermore, EPA uses toxicity as an indicator for chemical regulation. Also, toxicity profile is a key for a successful drug discovery. However, prediction of the toxicity is a difficult computation problem.

Hence, there is a need for an improved Artificial Intelligence (AI)-based computing system for predicting physical and chemical properties of eco-friendly materials, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an Artificial Intelligence (AI)-based computing system for predicting physical and chemical properties of eco-friendly materials is disclosed. The AI-based computing system includes one or more hardware processors and a memory coupled to the one or more hardware processors. The memory includes a plurality of modules in the form of programmable instructions executable by the one or more hardware processors. The plurality of modules include a data receiver module configured to receive Three-Dimensional (3D) structures and Density Function Theory (DFT) calculated energy data associated with one or more sesquioxides via one of: one or more electronic devices associated with a user and an external database. The plurality of modules include an energy prediction module configured to predict a formation energy of each of the one or more sesquioxides based on the received 3D structures and the DFT calculated energy data by using a property prediction-based AI model. Further, the energy prediction module is configured to predict a bandgap energy of each of the one or more sesquioxides based on the received 3D structures and the DFT calculated energy data by using the property prediction-based AI model upon predicting the formation energy. The plurality of modules also include a material determination module configured to determine a best sesquioxide from the one or more sesquioxides based on the predicted formation energy, and the predicted bandgap energy by using the property prediction-based AI model. The plurality of modules include a data output module configured to output the predicted formation energy, the predicted bandgap energy, and the determined best sesquioxide on user interface screen of the one or more electronic devices associated with the user.

In accordance with another embodiment of the present disclosure, an AI-based method for predicting physical and chemical properties of eco-friendly materials is disclosed. The AI-based method includes receiving 3D structures and DFT calculated energy data associated with one or more sesquioxides via one of: one or more electronic devices associated with a user and an external database. The AI-based method includes predicting a formation energy of each of the one or more sesquioxides based on the received 3D structures and the DFT calculated energy data by using a property prediction-based AI model. Further, the AI-based method includes predicting a bandgap energy of each of the one or more sesquioxides based on the received 3D structures and the DFT calculated energy data by using a property prediction-based AI model upon predicting the formation energy. The AI-based method also includes determining a best sesquioxide from the one or more sesquioxides based on the predicted formation energy, and the predicted bandgap energy by using the property prediction-based AI model. Further, the AI-based method includes outputting the predicted formation energy, the predicted bandgap energy, and the determined best sesquioxide on user interface screen of the one or more electronic devices associated with the user.

Embodiment of the present disclosure also provide a non-transitory computer-readable storage medium having instructions stored therein that, when executed by a hardware processor, cause the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for predicting physical and chemical properties of eco-friendly materials, in accordance with an embodiment of the present disclosure;

FIG. 2 is a block diagram illustrating an exemplary Artificial Intelligence (AI)-based computing system for predicting physical and chemical properties of eco-friendly materials, in accordance with an embodiment of the present disclosure;

FIG. 3A-3D are block diagrams depicting an exemplary operation of the AI-based computing system for predicting physical and chemical properties of eco-friendly materials, in accordance with an embodiment of the present disclosure; and

FIG. 4 is a process flow diagram illustrating an exemplary AI-based method for predicting physical and chemical properties of eco-friendly materials, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

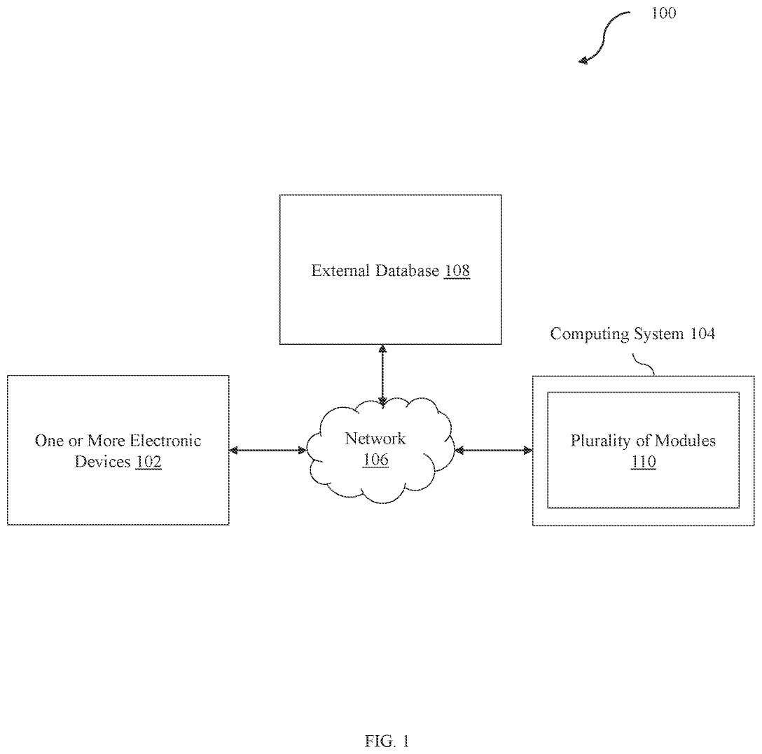

Referring now to the drawings, and more particularly to FIG. 1 through FIG. 4, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for predicting physical and chemical properties of eco-friendly materials, in accordance with an embodiment of the present disclosure. According to FIG. 1, the computing environment 100 includes one or more electronic devices 102 associated with a user communicatively coupled to an Artificial intelligence (AI)-based computing system 104 via a network 106. Further, the one or more electronic devices 102 are used by the user for providing Three-Dimensional (3D) structures and Density Function Theory (DFT) calculated energy data associated with one or more sesquioxides to the AI-based computing system 104. Furthermore, the one or more electronic devices 102 may also be used by the user to receive a predicted formation energy, a predicted bandgap energy, and a determined best sesquioxide. The AI-based computing system 104 may be hosted on a central server, such as cloud server or a remote server. In an embodiment of the present disclosure, the predicted formation energy of a molecular specie or a configuration is the energy required for (or released while) generating that configuration from molecules within a reference set. In an embodiment of the present disclosure, the bandgap energy is a gap in energy between a bound state and a free state, between a valence band and a conduction band. Further, the network 106 may be a Wireless-Fidelity (Wi-Fi) connection, a hotspot connection, a Bluetooth connection, a local area network, a wide area network or any other wireless network. In an exemplary embodiment of the present disclosure, the one or more electronic devices 102 may include a laptop computer, desktop computer, tablet computer, smartphone, wearable device, smart watch, and the like.

Further, the computing environment 100 includes an external database 108 communicatively coupled to the AI-based computing system 104 via the network 106. The external database 108 stores the 3D structures, the DFT calculated energy data, a chemical data report, a biological data report, and the like. In an embodiment of the present disclosure, the external database 108 provides the 3D structures, the DFT calculated energy data, the chemical data report, the biological data report, and the like to the AI-based computing system 104.

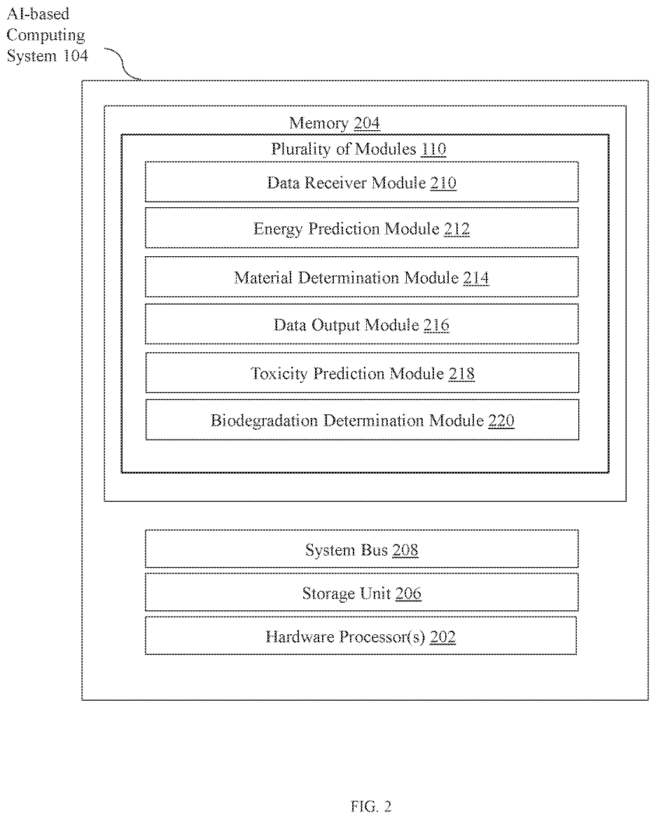

Furthermore, the one or more electronic devices 102 include a local browser, a mobile application or a combination thereof. Furthermore, the user may use a web application via the local browser, the mobile application, or a combination thereof to communicate with the AI-based computing system 104. In an embodiment of the present disclosure, the AI-based computing system 104 includes a plurality of modules 110. Details on the plurality of modules 110 have been elaborated in subsequent paragraphs of the present description with reference to FIG. 2.

In an embodiment of the present disclosure, the AI-based computing system 104 is configured to receive the 3D structures and the DFT calculated energy data associated with the one or more sesquioxides via the one or more electronic devices 102 associated with the user or the external database 108. The AI-based computing system 104 predicts the formation energy of each of the one or more sesquioxides based on the received 3D structures and the DFT calculated energy data by using a property prediction-based AI model. The AI-based computing system 104 predicts the bandgap energy of each of the one or more sesquioxides based on the received 3D structures and the DFT calculated energy data by using the property prediction-based AI model upon predicting the formation energy. Further, the AI-based computing system 104 determines a best sesquioxide from the one or more sesquioxides based on the predicted formation energy, and the predicted bandgap energy by using the property prediction-based AI model. The AI-based computing system 104 outputs the predicted formation energy, the predicted bandgap energy, and the determined best sesquioxide on user interface screen of the one or more electronic devices 102 associated with the user.

FIG. 2 is a block diagram illustrating an exemplary AI-based computing system 104 for predicting physical and chemical properties of eco-friendly materials, in accordance with an embodiment of the present disclosure. Further, the AI-based computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a data receiver module 210, an energy prediction module 212, a material determination module 214, a data output module 216, a toxicity prediction module 218, and a biodegradation determination module 220.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

The storage unit 206 may be a cloud storage, a Structured Query Language (SQL) data store or a location on a file system directly accessible by the plurality of modules 110. The storage unit 206 may store the 3D structures, the DFT calculated energy data, the formation energy, the bandgap energy, the best sesquioxide, a chemical data report, a biological data report, toxicity and non-toxicity of one or more molecules, a solvation free energy, degradable ability and non-degradable ability of each of the one or more molecules, a best soluble chemical, and the like.

The data receiver module 210 is configured to receive the 3D structures and the DFT calculated energy data associated with the one or more sesquioxides via the one or more electronic devices 102 associated with the user or the external database 108. In an exemplary embodiment of the present disclosure, the one or more sesquioxides include Aluminum sesquioxide, Gallium sesquioxide, Indium sesquioxide, and the like. In an exemplary embodiment of the present disclosure, the one or more electronic devices 102 may include a laptop computer, desktop computer, tablet computer, smartphone, wearable device, smart watch, and the like.

The energy prediction module 212 is configured to predict the formation energy of each of the one or more sesquioxides based on the received 3D structures and the DFT calculated energy data by using the property prediction-based AI model In an embodiment of the present disclosure, the predicted formation energy represents stability of each of the one or more sesquioxides. In an exemplary embodiment of the present disclosure, the property prediction-based AI model is a deep learning AI model. Further, the energy prediction module 212 predicts the bandgap energy of each of the one or more sesquioxides based on the received 3D structures and the DFT calculated energy data by using the property prediction-based AI model upon predicting the formation energy. In an embodiment of the present disclosure, the predicted bandgap energy represents conductivity of each of the one or more sesquioxides. For example, the prediction accuracy R² of the property prediction-based AI model for predicting the formation energy and the bandgap energy is 0.96.

The material determination module 214 is configured to determine the best sesquioxide from the one or more sesquioxides based on the predicted formation energy, and the predicted bandgap energy by using the property prediction-based AI model. In an embodiment of the present disclosure, the best sesquioxide has highest stability and conductivity amongst the one or more sesquioxides. In determining the best sesquioxide from the one or more sesquioxides based on the predicted formation energy, and the predicted bandgap energy by using the property prediction-based AI model, the material determination module 214 is configured to compare the predicted formation energy, and the predicted bandgap energy of one or more sesquioxides with each other by using the property prediction-based AI model. Further, the material determination module 214 determines the best sesquioxide from the one or more sesquioxides based on a result of comparison.

The data output module 216 is configured to output the predicted formation energy, the predicted bandgap energy, and the determined best sesquioxide on user interface screen of the one or more electronic devices 102 associated with the user.

In an embodiment of the present disclosure, the energy prediction module 212 receives a chemical data report of one or more chemicals via the one or more electronic devices 102 associated with the user or the external database 108. Further, the energy prediction module 212 predicts a solvation free energy of each of the one or more chemicals based on the received chemical data report by using the property prediction-based AI model. In an embodiment of the present disclosure, the solvation free energy is a reversible work required to insert a solute molecule into a solvent, at constant temperature and density (or pressure). In an embodiment of the present disclosure, the solvation energy represents solubility of each of the one or more chemicals. The solubility is an important molecular property considered for drug candidate and chemicals of other purpose. The energy prediction module 212 determines a best soluble chemical from the one or more chemicals based on the predicted solvation free energy by using the property prediction-based AI model. In determining the best soluble chemical from the one or more chemicals based on the predicted solvation free energy by using the property prediction-based AI model, the energy prediction module 212 compares the predicted solvation free energy of the one or more chemicals with each other by using the property prediction-based AI model. The energy prediction module 212 determines the best soluble chemical from the one or more chemicals based on a result of comparison. Furthermore, the energy prediction module 212 outputs the predicted solvation free energy, and the determined best soluble chemical on user interface screen of the one or more electronic devices 102 associated with the user. For example, the prediction accuracy R² of the property prediction-based AI model for predicting the solvation free energy is 0.81.

The toxicity prediction module 218 receives a chemical data report, a biological data report or a combination thereof of the one or more molecules via the one or more electronic devices 102 associated with the user or the external database 108. Further, the toxicity prediction module 218 predicts toxicity, non-toxicity, or a combination thereof of each of the one or more molecules based on the received chemical data report, the received biological data report, or a combination thereof by using the property determination-based AI model. In an embodiment of the present disclosure, EPA uses predicted toxicity as an indicator for chemical regulation. Further, toxicity profile is a key for a successful drug discovery. The toxicity prediction module 218 classifies the one or more molecules based on the predicted toxicity, the predicted non-toxicity, or a combination thereof by using the property determination-based AI model. The toxicity prediction module 218 outputs the predicted toxicity, the predicted non-toxicity, a combination thereof, and the classified one or more molecules on user interface screen of the one or more electronic devices 102 associated with the user. For example, the prediction accuracy R² of the property prediction-based AI model for predicting the toxicity, the non-toxicity, or a combination thereof is 0.87.

The biodegradation determination module 220 is configured to receive the chemical data report, the biological data report of one or more molecules, or a combination thereof via the one or more electronic devices 102 associated with the user or the external database 108. The biodegradation determination module 220 determines degradable ability, non-degradable ability of each of the one or more molecules, or a combination thereof based on the received chemical data report, the received biological data report or a combination thereof by using the property determination-based AI model. In an embodiment of the present disclosure, biodegradation ability of a molecules is an important property in design environmental-friendly chemicals and materials. Further, the biodegradation determination module 220 classifies the one or more molecules based on the determined degradable ability, the determined non-degradable ability or a combination thereof by using the property determination-based AI model. The biodegradation determination module 220 outputs the determined degradable ability, the determined non-degradable ability, or a combination thereof, and the classified one or more molecules on user interface screen of the one or more electronic devices 102 associated with the user. For example, the prediction accuracy R² of the property prediction-based AI model for predicting degradable ability, non-degradable ability of each of the one or more molecules, or a combination thereof is 0.96.

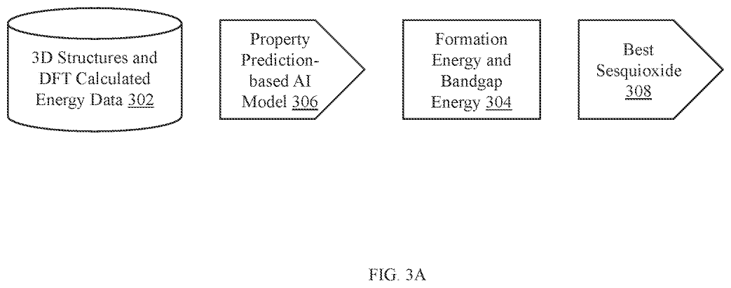

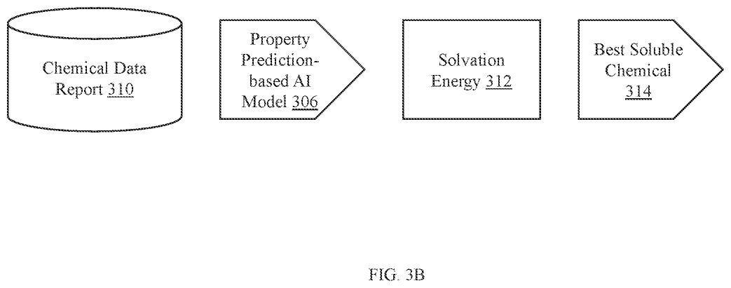

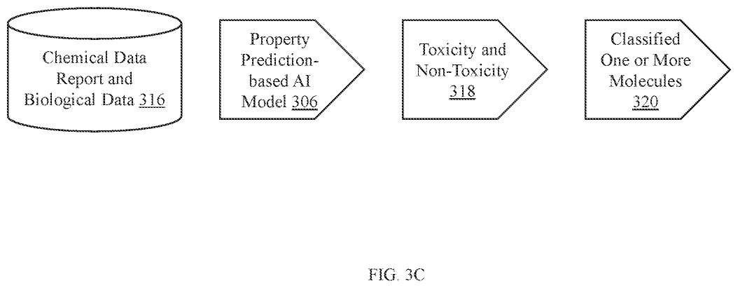

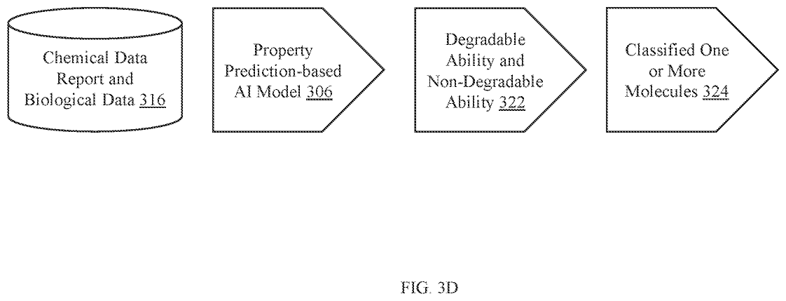

FIG. 3A-3D are block diagrams depicting an exemplary operation of the AI-based computing system 104 for predicting physical and chemical properties of eco-friendly materials, in accordance with an embodiment of the present disclosure. The AI-based computing system 104 receives the 3D structures and the DFT calculated energy data 302 associated with the one or more sesquioxides via the one or more electronic devices 102 associated with the user or the external database 108. Further, the AI-based computing system 104 predicts the formation energy and the bandgap energy 304 of each of the one or more sesquioxides based on the received 3D structures and the DFT calculated energy data 302 by using the property prediction-based AI model 306. At step 308, the AI-based computing system 104 determines the best sesquioxide from the one or more sesquioxides based on the predicted formation energy, and the predicted bandgap energy 304 by using the property prediction-based AI model 306.

Further, the AI-based computing system 104 receives the chemical data report 310 of the one or more chemicals via the one or more electronic devices 102 associated with the user or the external database 108. The AI-based computing system 104 predicts the solvation free energy 312 of each of the one or more chemicals based on the received chemical data report 310 by using the property prediction-based AI model 306. At step 314, the AI-based computing system 104 determines the best soluble chemical from the one or more chemicals based on the predicted solvation free energy 312 by using the property prediction-based AI model 306. Furthermore, the AI-based computing system 104 receives the chemical data report, the biological data report of one or more molecules or a combination thereof 316 via the one or more electronic devices 102 associated with the user or the external database 108. The AI-based computing system 104 predicts the toxicity, the non-toxicity of each of the one or more molecules or a combination thereof 318 based on the received chemical data report, the received biological data report or a combination thereof 316 by using the property determination-based AI model 306. At step 320, the AI-based computing system 104 classifies the one or more molecules based on the predicted toxicity, the predicted non-toxicity or a combination thereof by using the property determination-based AI model 306.

Furthermore, the AI-based computing system 104 receives the chemical data report, the biological data report of one or more molecules or a combination thereof 316 via the one or more electronic devices 102 associated with the user or the external database 108. The AI-based computing system 104 determines degradable ability, non-degradable ability or a combination thereof 322 of each of the one or more molecules based on the received chemical data report, the received biological data report or a combination thereof 316 by using the property determination-based AI model 306. At step 324, the AI-based computing system 104 classifies the one or more molecules based on the determined degradable ability, the determined non-degradable ability or a combination thereof 322 by using the property determination-based AI model 306.

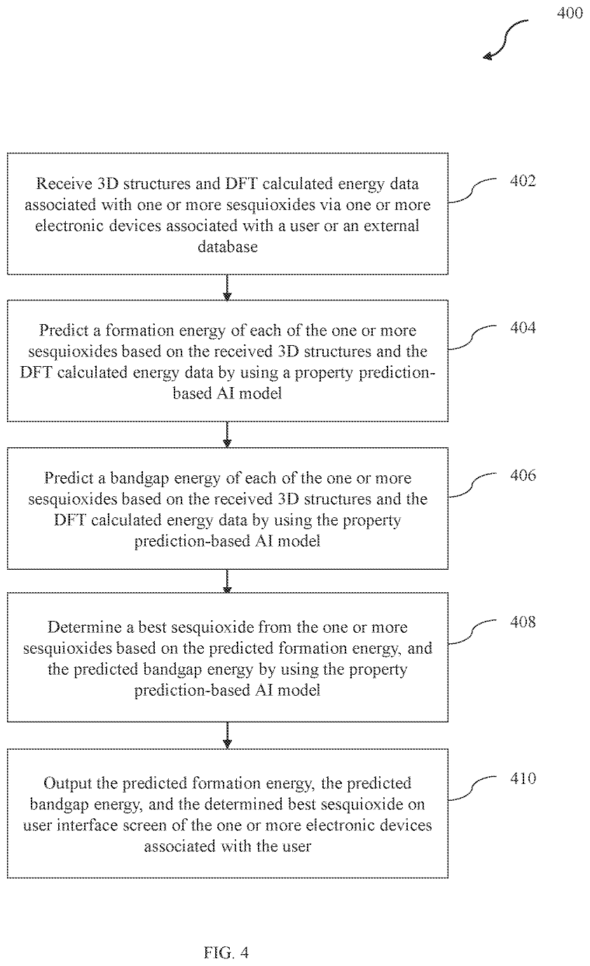

FIG. 4 is a process flow diagram illustrating an exemplary AI-based method for predicting physical and chemical properties of eco-friendly materials, in accordance with an embodiment of the present disclosure. At step 402, 3D structures and DFT calculated energy data associated with one or more sesquioxides are received via one or more electronic devices 102 associated with a user or an external database 108. In an exemplary embodiment of the present disclosure, the one or more sesquioxides include Aluminum sesquioxide, Gallium sesquioxide, Indium sesquioxide, and the like. In an exemplary embodiment of the present disclosure, the one or more electronic devices 102 may include a laptop computer, desktop computer, tablet computer, smartphone, wearable device, smart watch, and the like.

At step 404, formation energy of each of the one or more sesquioxides is predicted based on the received 3D structures and the DFT calculated energy data by using a property prediction-based AI model. In an embodiment of the present disclosure, the predicted formation energy represents stability of each of the one or more sesquioxides. In an exemplary embodiment of the present disclosure, the property prediction-based AI model is a deep learning AI model.

At step 406, a bandgap energy of each of the one or more sesquioxides is predicted based on the received 3D structures and the DFT calculated energy data by using the property prediction-based AI model upon predicting the formation energy. In an embodiment of the present disclosure, the predicted bandgap energy represents conductivity of each of the one or more sesquioxides. For example, the prediction accuracy R² of the property prediction-based AI model for predicting the formation energy and the bandgap energy is 0.96.

At step 408, a best sesquioxide is determined from the one or more sesquioxides based on the predicted formation energy, and the predicted bandgap energy by using the property prediction-based AI model. In an embodiment of the present disclosure, the best sesquioxide has highest stability and conductivity amongst the one or more sesquioxides.

In determining the best sesquioxide from the one or more sesquioxides based on the predicted formation energy, and the predicted bandgap energy by using the property prediction-based AI model, the AI-based method 400 includes comparing the predicted formation energy, and the predicted bandgap energy of one or more sesquioxides with each other by using the property prediction-based AI model. Further, the AI-based method 400 includes determining the best sesquioxide from the one or more sesquioxides based on a result of comparison.

At step 410, the predicted formation energy, the predicted bandgap energy, and the determined best sesquioxide are outputted on user interface screen of the one or more electronic devices 102 associated with the user.

In an embodiment of the present disclosure, the AI-based method 400 includes receiving a chemical data report of one or more chemicals via the one or more electronic devices 102 associated with the user or the external database 108. Further, the AI-based method 400 includes predicting a solvation free energy of each of the one or more chemicals based on the received chemical data report by using the property prediction-based AI model. In an embodiment of the present disclosure, the solvation free energy is a reversible work required to insert a solute molecule into a solvent, at constant temperature and density (or pressure). In an embodiment of the present disclosure, the solvation energy represents solubility of each of the one or more chemicals. The solubility is an important molecular property considered for drug candidate and chemicals of other purpose. The AI-based method 400 includes determining a best soluble chemical from the one or more chemicals based on the predicted solvation free energy by using the property prediction-based AI model. In determining the best soluble chemical from the one or more chemicals based on the predicted solvation free energy by using the property prediction-based AI model, the AI-based method 400 includes comparing the predicted solvation free energy of the one or more chemicals with each other by using the property prediction-based AI model. The AI-based method 400 includes determining the best soluble chemical from the one or more chemicals based on a result of comparison. Furthermore, the AI-based method 400 includes outputting the predicted solvation free energy, and the determined best soluble chemical on user interface screen of the one or more electronic devices 102 associated with the user. For example, the prediction accuracy R² of the property prediction-based AI model for predicting the solvation free energy is 0.81.

Further, the AI-based method 400 includes receiving a chemical data report, a biological data report or a combination thereof of the one or more molecules via the one or more electronic devices 102 associated with the user or the external database 108. Further, AI-based method 400 includes predicting toxicity, non-toxicity, or a combination thereof of each of the one or more molecules based on the received chemical data report, the received biological data report, or a combination thereof by using the property determination-based AI model. In an embodiment of the present disclosure, EPA uses predicted toxicity as an indicator for chemical regulation. Further, toxicity profile is a key for a successful drug discovery. The AI-based method 400 includes classifying the one or more molecules based on the predicted toxicity, the predicted non-toxicity, or a combination thereof by using the property determination-based AI model. The AI-based method 400 includes outputting the predicted toxicity, the predicted non-toxicity, a combination thereof, and the classified one or more molecules on user interface screen of the one or more electronic devices 102 associated with the user. For example, the prediction accuracy R² of the property prediction-based AI model for predicting the toxicity, the non-toxicity, or a combination thereof is 0.87.

Furthermore, the AI-based method 400 includes receiving the chemical data report, the biological data report of one or more molecules, or a combination thereof via the one or more electronic devices 102 associated with the user or the external database 108. The AI-based method 400 includes determining degradable ability, non-degradable ability of each of the one or more molecules, or a combination thereof based on the received chemical data report, the received biological data report or a combination thereof by using the property determination-based AI model. In an embodiment of the present disclosure, biodegradation ability of a molecules is an important property in design environmental-friendly chemicals and materials. Further, the AI-based method 400 includes classifying the one or more molecules based on the determined degradable ability, the determined non-degradable ability or a combination thereof by using the property determination-based AI model. The AI-based method 400 includes outputting the determined degradable ability, the determined non-degradable ability, or a combination thereof, and the classified one or more molecules on user interface screen of the one or more electronic devices 102 associated with the user. For example, the prediction accuracy R² of the property prediction-based AI model for predicting degradable ability, non-degradable ability of each of the one or more molecules, or a combination thereof is 0.96.

The method 400 may be implemented in any suitable hardware, software, firmware, or combination thereof.

Thus, various embodiments of the present AI-based computing system 104 provide a solution to predict physical and chemical properties of eco-friendly materials. In an embodiment of the present disclosure, the AI-based computing system 104 determines the best sesquioxide from the one or more sesquioxides based on the predicted formation energy, and the predicted bandgap energy by using the property prediction-based AI model. Since prediction accuracy R² of the predicted formation energy, and the predicted bandgap energy is 0.96, the AI-based computing system 104 accurately determines the best sesquioxide from the one or more sesquioxides. Further, the AI-based computing system 104 determines the best soluble chemical from the one or more chemicals based on the predicted solvation free energy. In an embodiment of the present disclosure, the AI-based computing system 104 is hosted on the central server, such as cloud server or a remote server, to achieve elastic storage and computing solution at the backend. The AI-based computing system 104 also allows data expansion and integration to billions of molecules. Furthermore, the AI-based computing system 104 achieves application expansion up to hundreds of physical, chemical, biological property profiles, or a any combination thereof. The property determination-based AI model is a reinforcement model which continuously learns and re-train based on past actions.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (I/O) adapter. The/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

1. An Artificial Intelligence (AI)-based computing system for predicting physical and chemical properties of eco-friendly materials, the AI-based computing system comprising:
one or more hardware processors; and
a memory coupled to the one or more hardware processors, wherein the memory comprises a plurality of modules in the form of programmable instructions executable by the one or more hardware processors, and wherein the plurality of modules comprises:
a data receiver module configured to receive Three-Dimensional (3D) structures and Density Function Theory (DFT) calculated energy data associated with one or more sesquioxides via one of: one or more electronic devices associated with a user and an external database;
an energy prediction module configured to:
predict a formation energy of each of the one or more sesquioxides based on the received 3D structures and the DFT calculated energy data by using a property prediction-based AI model;
predict a bandgap energy of each of the one or more sesquioxides based on the received 3D structures and the DFT calculated energy data by using the property prediction-based AI model upon predicting the formation energy;
a material determination module configured to determine a best sesquioxide from the one or more sesquioxides based on the predicted formation energy, and the predicted bandgap energy by using the property prediction-based AI model; and
a data output module configured to output the predicted formation energy, the predicted bandgap energy, and the determined best sesquioxide on user interface screen of the one or more electronic devices associated with the user.

2. The AI-based computing system of claim 1, wherein in determining the best sesquioxide from the one or more sesquioxides based on the predicted formation energy, and the predicted bandgap energy by using the property prediction-based AI model, the material determination module is configured to:
compare the predicted formation energy, and the predicted bandgap energy of one or more sesquioxides with each other by using the property prediction-based AI model; and
determine the best sesquioxide from the one or more sesquioxides based on a result of comparison.

3. The AI-based computing system of claim 1, wherein the one or more sesquioxides comprise at least one of Aluminum sesquioxide, Gallium sesquioxide, and Indium sesquioxide.

4. The AI-based computing system of claim 1, wherein the best sesquioxide has highest stability and conductivity amongst the one or more sesquioxides.

5. The AI-based computing system of claim 1, wherein the energy prediction module is configured to:
receive a chemical data report of one or more chemicals via one of: the one or more electronic devices associated with the user and the external database;
predict a solvation free energy of each of the one or more chemicals based on the received chemical data report by using the property prediction-based AI model;
determine a best soluble chemical from the one or more chemicals based on the predicted solvation free energy by using the property prediction-based AI model; and
output the predicted solvation free energy, and the determined best soluble chemical on user interface screen of the one or more electronic devices associated with the user.

6. The AI-based computing system of claim 5, wherein in determining the best soluble chemical from the one or more chemicals based on the predicted solvation free energy by using the property prediction-based AI model, the energy prediction module is configured to:
compare the predicted solvation free energy of the one or more chemicals with each other by using the property prediction-based AI model; and
determine the best soluble chemical from the one or more chemicals based on a result of comparison.

7. The AI-based computing system of claim 1, further comprising a toxicity prediction module configured to:
receive at least one of: a chemical data report and a biological data report of one or more molecules via one of: the one or more electronic devices associated with the user and the external database;
predict at least one of: toxicity and non-toxicity of each of the one or more molecules based on the received at least one of the chemical data report and the biological data report by using the property determination-based AI model;
classify the one or more molecules based on the predicted at least one of: toxicity and non-toxicity by using the property determination-based AI model; and
output the predicted at least one of: toxicity and non-toxicity, and the classified one or more molecules on user interface screen of the one or more electronic devices associated with the user.

8. The AI-based computing system of claim 1, further comprising a biodegradation determination module configured to:
receive at least one of: a chemical data report and a biological data report of one or more molecules via one of: the one or more electronic devices associated with the user and the external database;
determine at least one of: degradable ability and non-degradable ability of each of the one or more molecules based on the received at least one of: the chemical data report and the biological data report by using the property determination-based AT model;
classify the one or more molecules based on the determined at least one of: degradable ability and non-degradable ability by using the property determination-based AI model, and
output the determined at least one of: degradable ability and non-degradable ability, and the classified one or more molecules on user interface screen of the one or more electronic devices associated with the user.

9. An Artificial Intelligence (AI)-based method for predicting physical and chemical properties of eco-friendly materials, the AI-based method comprising:
receiving, by one or more hardware processors, Three-Dimensional (3D) structures and Density Function Theory (DFT) calculated energy data associated with one or more sesquioxides via one of: one or more electronic devices associated with a user and an external database;
predicting, by the one or more hardware processors, a formation energy of each of the one or more sesquioxides based on the received 3D structures and the DFT calculated energy data by using a property prediction-based AI model;
predicting, by the one or more hardware processors, a bandgap energy of each of the one or more sesquioxides based on the received 3D structures and the DFT calculated energy data by using the property prediction-based AI model upon predicting the formation energy;
determining, by the one or more hardware processors, a best sesquioxide from the one or more sesquioxides based on the predicted formation energy, and the predicted bandgap energy by using the property prediction-based AI model; and
outputting, by the one or more hardware processors, the predicted formation energy, the predicted bandgap energy, and the determined best sesquioxide on user interface screen of the one or more electronic devices associated with the user.

10. The AI-based method of claim 9, wherein determining the best sesquioxide from the one or more sesquioxides based on the predicted formation energy, and the predicted bandgap energy by using the property prediction-based AI model comprises
comparing the predicted formation energy, and the predicted bandgap energy of one or more sesquioxides with each other by using the property prediction-based AI model; and
determining the best sesquioxide from the one or more sesquioxides based on a result of comparison.

11. The AI-based method of claim 9, wherein the one or more sesquioxides comprise at least one of Aluminum sesquioxide, Gallium sesquioxide, and Indium sesquioxide.

12. The AI-based method of claim 9, wherein the best sesquioxide has highest stability and conductivity amongst the one or more sesquioxides.

13. The AI-based method of claim 9, further comprising:
receiving a chemical data report of one or more chemicals via one of: the one or more electronic devices associated with the user and the external database;
predicting a solvation free energy of each of the one or more chemicals based on the received chemical data report by using the property prediction-based AI model;
determining a best soluble chemical from the one or more chemicals based on the predicted solvation free energy by using the property prediction-based AI model; and
outputting the predicted solvation free energy, and the determined best soluble chemical on user interface screen of the one or more electronic devices associated with the user.

14. The AI-based method of claim 13, wherein determining the best soluble chemical from the one or more chemicals based on the predicted solvation free energy by using the property prediction-based AI model comprises:
comparing the predicted solvation free energy of the one or more chemicals with each other by using the property prediction-based AI model; and
determining the best soluble chemical from the one or more chemicals based on a result of comparison.

15. The AI-based method of claim 9, further comprising:
receiving at least one of: a chemical data report and a biological data report of one or more molecules via one of: the one or more electronic devices associated with the user and the external database;
predicting at least one of: toxicity and non-toxicity of each of the one or more molecules based on the received at least one of: the chemical data report and the biological data report by using the property determination-based AI model;
classifying the one or more molecules based on the predicted at least one of: toxicity and non-toxicity by using the property determination-based AI model; and
outputting the predicted at least one of: toxicity and non-toxicity, and the classified one or more molecules on user interface screen of the one or more electronic devices associated with the user

16. The AI-based method of claim 9, further comprising:
receiving at least one of: a chemical data report and a biological data report of one or more molecules via one of: the one or more electronic devices associated with the user and the external database;
determining at least one of: degradable ability and non-degradable ability of each of the one or more molecules based on the received at least one of: the chemical data report and the biological data report by using the property determination-based AI model;
classifying the one or more molecules based on the determined at least one of: degradable ability and non-degradable ability by using the property determination-based AI model; and
outputting the determined at least one of: degradable ability and non-degradable ability, and the classified one or more molecules on user interface screen of the one or more electronic devices associated with the user.

17. A non-transitory computer-readable storage medium having instructions stored therein that, when executed by a hardware processor, cause the processor to perform method steps comprising:
receiving Three-Dimensional (3D) structures and Density Function Theory (DFT) calculated energy data associated with one or more sesquioxides via one of: one or more electronic devices associated with a user and an external database;
predicting a formation energy of each of the one or more sesquioxides based on the received 3D structures and the DFT calculated energy data by using a property prediction-based AI model;
predicting a bandgap energy of each of the one or more sesquioxides based on the received 3D structures and the DFT calculated energy data by using the property prediction-based AI model upon predicting the formation energy;
determining a best sesquioxide from the one or more sesquioxides based on the predicted formation energy, and the predicted bandgap energy by using the property prediction-based AI model; and
outputting the predicted formation energy, the predicted bandgap energy, and the determined best sesquioxide on user interface screen of the one or more electronic devices associated with the user.

18. The non-transitory computer-readable storage medium of claim 17, wherein detecting the best sesquioxide from the one or more sesquioxides based on the determined formation energy, and the determined bandgap energy by using the property determination-based AI model comprises:
comparing the predicted formation energy, and the predicted bandgap energy of one or more sesquioxides with each other by using the property prediction-based AI model; and
determining the best sesquioxide from the one or more sesquioxides based on a result of comparison.

19. The non-transitory computer-readable storage medium of claim 17, wherein the one or more sesquioxides comprise at least one of Aluminum sesquioxide, Gallium sesquioxide, and Indium sesquioxide.

20. The non-transitory computer-readable storage medium of claim 17, wherein the best sesquioxide has highest stability and conductivity amongst the one or more sesquioxides.
